(19) United States
(12) Patent Application Publication
(10) Pub. No.: US 2016/0222367 A1
(43) Pub. Date: Aug. 4, 2016

(54) ENZYME COMPOSITION AND PROCESS OF PREPARATION THEREOF

(71) Applicant: COUNCIL OF SCIENTIFIC & INDUSTRIAL RESEARCH, New Delhi (IN)

(72) Inventors: Jayant Malhar Khire, Pune (IN); Pradnya Deepak Gujar, Pune (IN)

(21) Appl. No.: 14/916,763

(22) PCT Filed: Sep. 5, 2014
(86) PCT No.: PCT/IN2014/000583

§ 371 Date: Mar. 4, 2016

(30) Foreign Application Priority Data
Sep. 6, 2013 (IN) 2641/DEL/2013

(51) Int. Cl. C12N 9/24; C12N 9/38; C12N 9/42; C12N 9/30; C12N 9/34

(52) U.S. Cl. CPC C12N 9/2482; C12N 9/242; C12N 9/2428; C12N 9/2437; C12N 9/2471; C12Y 302/01001; C12Y 302/01003; C12Y 302/01004; C12Y 302/01015; C12Y 302/01023; C12Y 302/01008

(57) ABSTRACT

The present disclosure provides an enzyme composition comprising CMCase, xylanase, acid phosphatase, amylase, phytase, β-glucosidase, pectinase and α-galactosidase. More specifically, the present disclosure provides a process for the synthesis of an enzyme composition from Aspergillus niger MTCC5231, isolated from soil and its mutant strains Aspergillus niger.

### FIELD OF INVENTION

The present disclosure relates to an enzyme composition. More specifically, the present disclosure relates to a process for the synthesis of an enzyme composition from Aspergillus niger NCIM 563 (MTCC 5213), isolated from soil and its mutant strains A. niger MCC0013 and MCC0014.

### BACKGROUND OF THE INVENTION

Phytase has low activity under submerged fermentation conditions. The inventors have published that the organism Aspergillus niger can produce mutant strains and that both isolate and mutants produce phytase enzyme (Bhaysar el al., Appl Microbiol Biotechnol., 2013, 97(2), 673-9. Hence, the mutants of Aspergillus niger NCIM 563 overproducing phytase under such conditions have direct commercial potential.

Mutant strains show higher activity of phytase than earlier reports. New more efficient strains generated using combination of UV and Chemical mutagenesis.

### OBJECTIVE OF THE INVENTION

The main object of the present disclosure is to provide a process for the synthesis of an enzyme composition from Aspergillus niger NCIM 563, isolated from soil and its mutant strains Aspergillus niger MCC0013 and MCC0014.

### SUMMARY OF THE INVENTION

In an aspect of the present disclosure, there is provided a process for the production of multienzyme system using fermentation, said process comprising: (a) growing Aspergillus niger on a medium having sucrose at a concentration of about 1% and yeast extract at a concentration of about 0.2% and at a temperature in the range of 25-35 deg C. and pH in the range of 5-6 for a period in the range of 48-120 hours under shaking condition having angular speed in the range of 200-250 rpm in order to obtain an inoculum; (b) adding the inoculum, obtained in step (a), to the fermentation medium at the concentration in the range of 10-25 percent (v/v) followed by fermenting for a period in the range of 48-120 hours under shaking condition having angular speed in the range of 200-250 rpm for a period in the range of 48-120 hours in order to obtain the fermentation culture; wherein said fermentation medium has the following composition:

| Ingredients | Proportion (percent) |
|---|---|
| starch | 3-5 |
| glucose | 0.5-1.5 |
| carboxy methyl cellulose | 0.5-1.5 |
| pectin | 0.5-1.5 |
| yeast extract | 0.6-1.0 |
| KH₂PO₄ | 0.7-1.1 |
| NaNO₃ | 1-3 |
| MgSO₄•7H₂0 | 0.02-0.04 |
| K₂HPO₄ | 0.2-0.6 |
| NaC1 | 0.4-0.8 |
| NH₄C1 | 0.8-1.2 |

and, (c) centrifuging the fermentation culture, obtained in step (b), followed by separation of culture fluid from pellets using filter paper (Whatman no.1) to obtain the said multienzyme system in form of culture fluids.

### DETAILED DESCRIPTION OF THE INVENTION

In an embodiment of the present disclosure, there is provided a process for the production of multienzyme system using fermentation, said process comprising: (a) growing Aspergillus niger on a medium having sucrose at a concentration of about 1% and yeast extract at a concentration of about 0.2% and at a temperature in the range of 25-35 deg C. and pH in the range of 5-6 for a period in the range of 48-120 hours under shaking condition having angular speed in the range of 200-250 rpm in order to obtain an inoculum; (b) adding the inoculum, obtained in step (a), to the fermentation medium at the concentration in the range of 10-25 percent(v/v) followed by fermenting for a period in the range of 48-120 hours under shaking condition having angular speed in the range of 200-250 rpm for a period in the range of 48-120 hours in order to obtain the fermentation culture; wherein said fermentation medium has the following composition:

| Ingredients | Proportion (percent) |
|---|---|
| starch | 3-5 |
| glucose | 0.5-1.5 |
| carboxy methyl cellulose | 0.5-1.5 |
| pectin | 0.5-1.5 |
| yeast extract | 0.6-1.0 |
| KH₂PO₄ | 0.7-1.1 |
| NaNO₃ | 1-3 |
| MgSO₄•7H₂0 | 0.02-0.04 |
| K₂HPO₄ | 0.2-0.6 |
| NaC1 | 0.4-0.8 |
| NH₄C1 | 0.8-1.2 |

and, (c) centrifuging the fermentation culture, obtained in step (b), followed by separation of culture fluid from pellets using filter paper (Whatman no.1) to obtain the said multienzyme system in form of culture fluids.

In an embodiment of the present disclosure, there is provided an enzyme composition from Aspergillus niger NCIM 563 Aspergillus niger NCIM 563: NCIM NRS-2 (1953). Putman strain 3, isolated from soil and its mutant strains. A. niger MCC0013 and MCC0014.

In an embodiment of the present disclosure, there is provided an enzyme composition comprising CMCase, xylanase, acid phosphatase, amylase, phytase, β-glucosidase, pectinase and α-galactosidase.

In an embodiment of the present disclosure, there is provided an isolated fungal strain Aspergillus niger NCIM 563 deposited with Microbial Type Culture Collection and Gene Bank (MTCC) having accession number MTCC 5213.

In an embodiment, the present disclosure, there is provided a process for the synthesis of an enzyme composition comprising CMCase, xylanase, acid phosphatase, amylase, Phytase, β-glucosidase, pectinase and α-galactosidase from A. niger NCIM 563, isolated from soil and its mutant strains A. niger MCC0013 and MCC0014, said process being a submerged fermentation process comprising the steps of:
a. Sterilizing the fermentation medium by autoclaving at a temperature in the range of 110 to 140 ° C. for 15 min;
b. Cooling the fermentation medium of step (a) followed by inoculating the medium with desired spore suspension(1 ml of spore suspension containing 1×10⁸ spores/ml) and adjusting the incubation time as per the experimental design;
c. Incubating flasks were at 30 ° C. at 200 rpm for 4-11 days afforded the desired enzyme composition.

In an embodiment, the present disclosure, there is provided an enzyme composition obtained from Aspergillus niger NCIM 563, said composition comprising:
a. CMCase having activity in the range of 0.04 to 0.17 IU/ml;
b. β-glucosidase having activity in the range of 0.52 to 2.04 IU/ml;
c. acid phosphatase having activity in the range of 50.68 to 197.66 IU/ml;
d. Amylase having activity in the range of 1.9 to 7.41 IU/ml;
e. Phytase having activity in the range of 17.53 to 68.30 IU/ml;

In another embodiment of the present disclosure, there is provided an enzyme composition obtained from Aspergillus niger mutant NCIM MCC0014, said composition comprising:
a. CMCase having activity in the range of 0.02 to 0.1 IU/ml;
b. Xylanase having activity in the range of 0.05 to 0.19 IU/ml;
c. β-glucosidase having activity in the range of 0.39 to 1.60 IU/ml;
d. Acid phosphatase having activity in the range of 13.08 to 53.36 IU/ml;
e. Amylase having activity in the range of 0.59 to 2.41 IU/ml;
f. Phytase having activity in the range of 38.38 to 156.95 IU/ml;

In yet another embodiment, the present invention provides an enzyme composition obtained from Aspergillus niger mutant NCIM MCC0014, said composition comprising:
a. CMCase having activity in the range of 0.07 to 0.16 1U/ml;
b. Xylanase having activity in the range of 0.15 to 0.34 IU/ml;
c. β-glucosidase having activity in the range of 0.02 to 0.05 IU/ml;
d. Acid phosphatase having activity in the range of 25.88 to 57.19 IU/ml;
e. Amylase having activity in the range of 1.42 to 3.14 IU/ml;
f. Phytase having activity in the range of 43.22 to 67.51 IU/ml;

### EXAMPLES

The following examples are given by way of illustration of the present invention and therefore should not be construed to limit the scope of the present invention.

### Example 1

### Materials and Methods

### Chemicals

Phytic acid sodium salt was purchased from Sigma Chemical Company (St. Louis, Mo., USA). All other chemicals used were of analytical grade. Rice bran was purchased from the local market.

Microorganism, culture media, and enzyme production

Aspergillus niger MTCC 5213, used in the present disclosure, was maintained on Potato Dextrose Agar (PDA) slant and stored at 4° C. Spores for inoculation were obtained by culturing the strain at 30° C. on a PDA slant for 7 days, followed by washing with 10 ml sterile saline containing 0.01% Tween 80.

The basal fermentation medium according to Bhaysar et al. 2008 contained rice bran, glucose, NaNO₃, MgSO₄.7H₂O, KCl, and FeSO₄.7H₂O. The fermentation medium for optimization via statistical design of experiments included additional components, namely, MnSO₄, dextrin, and Tween 80 at various concentrations as required by the experimental design.

The fermentation medium pH 5.5 before sterilization (100 ml in 250 ml Erlenmeyer flask) was sterilized by autoclaving at 121 ° C. for 15 min. On cooling the fermentation medium was inoculated with desired spore suspension and the incubation time adjusted as per the experimental design. Flasks were incubated at 30° C. at 200 rpm and samples removed after every 24 h. Enzyme production was expressed as enzyme activity U/ml.

### Partial Purification of Enzyme

After fermentation, the mycelium was separated by filtration followed by centrifugation at 10,000×g for 30 min and the clear supernatant was collected. Solid ammonium sulphate was added to the supernatant to 95% saturation with constant stirring. The precipitate was collected by centrifugation at 15,000×g for 20 min and dissolved in minimum volume of 100 mM Glycine-HCl buffer, pH 2.5 and the salt was removed by passing through Sephadex G-25 column. Active fractions were concentrated through YM-30 membrane (Millipore) and used for enzyme activity measurement.

### Example 2

### Production of Different Hydrolytic Enzymes

Parent 563

| | Day | Day | Day | Day | Range of Activity |
|---|---|---|---|---|---|
| | 4 | 7 | 10 | 11 | (IU/ml) |
| CMCase (IU/ml) | 0.04 | 0.08 | 0.13 | 0.17 | 0.04 to 0.17 |
| Xylanase (IU/ml) | 0.00 | 0.00 | 0.00 | 0.00 | 0 |
| β-glucosidase | 0.52 | 0.91 | 1.49 | 2.04 | 0.52 to 2.04 |
| (IU/ml) | | | | | |
| Acid Phosphatase | 50.68 | 88.24 | 144.28 | 197.66 | 50.68 to 197.66 |
| (IU/ml) | | | | | |
| Amylase (IU/ml) | 1.90 | 3.31 | 5.41 | 7.41 | 1.9 to 7.41 |
| Phytase (IU/ml) | 17.53 | 30.52 | 49.99 | 68.30 | 17.53 to 68.30 |

Mutant MCC0013

| | Day | Day | Day | Day | Range of Activity |
|---|---|---|---|---|---|
| | 4 | 7 | 10 | 11 | (IU/ml) |
| CMCase (IU/ml) | 0.02 | 0.05 | 0.07 | 0.10 | 0.02 to 0.1 |
| Xylanase (IU/ml) | 0.05 | 0.10 | 0.14 | 0.19 | 0.05 to 0.19 |
| β-glucosidase | 0.39 | 0.87 | 1.23 | 1.60 | 0.39 to 1.60 |
| (IU/ml) | | | | | |
| Acid Phosphatase | 13.08 | 29.16 | 41.05 | 53.36 | 13.08 to 53.36 |
| (IU/ml) | | | | | |
| Amylase (IU/ml) | 0.59 | 1.32 | 1.85 | 2.41 | 0.59 to 2.41 |
| Phytase (IU/ml) | 38.38 | 86.00 | 121.21 | 156.95 | 38.38 to 156.95 |

Mutant MCC0014

| | Day | Day | Day | Day | Range of Activity |
|---|---|---|---|---|---|
| | 4 | 7 | 10 | 11 | (IU/ml) |
| CMCase (IU/ml) | 0.07 | 0.16 | 0.11 | NA | 0.07 to 0.16 |
| Xylanase (IU/ml) | 0.15 | 0.34 | 0.24 | NA | 0.15 to 0.34 |
| β-glucosidase | 0.02 | 0.05 | 0.04 | NA | 0.02 to 0.05 |
| (IU/ml) | | | | | |
| Acid Phosphatase | 25.88 | 57.19 | 40.56 | NA | 25.88 to 57.19 |
| (IU/ml) | | | | | |
| Amylase (IU/ml) | 1.42 | 3.14 | 2.23 | NA | 1.42 to 3.14 |
| Phytase (IU/ml) | 43.22 | 95.46 | 67.51 | NA | 43.22 to 67.51 |

### ADVANTAGES OF THE INVENTION

a. Wider enzyme activities of Aspergillus niger.
b. Reduction in fermentation time for mutant NCIM MCC0014 by 7 days and for mutant NCIM MCC0013 by 1 day.
c. Along with increased activity these organisms also show morphological variations under scanning electron microscope. Additionally the activity profile of other enzymes of the mutants and parent culture are different.

I/we claim:

1. A process for the production of multienzyme system using fermentation, said process comprising:
a) growing Aspergillus niger on a medium having sucrose at a concentration of about 1% and yeast extract at a concentration of about 0.2% and at a temperature in the range of 25-35 deg C. and pH in the range of 5-6 for a period in the range of 48-120 hours under shaking condition having angular speed in the range of 200-250 rpm in order to obtain an inoculum;
b) adding the inoculum, obtained in step (a), to the fermentation medium at the concentration in the range of 10-25 percent (v/v) followed by fermenting for a period in the range of 48-120 hours under shaking condition having angular speed in the range of 200-250 rpm for a period in the range of 48-120 hours in order to obtain the fermentation culture; wherein said fermentation medium has the following composition:

| Ingredients | Proportion (percent) |
|---|---|
| starch | 3-5 |
| glucose | 0.5-1.5 |
| carboxy methyl cellulose | 0.5-1.5 |
| pectin | 0.5-1.5 |
| yeast extract | 0.6-1.0 |
| KH₂PO₄ | 0.7-1.1 |
| NaNO₃ | 1-3 |
| MgSO₄•7H₂0 | 0.02-0.04 |
| K₂HPO₄ | 0.2-0.6 |
| NaC1 | 0.4-0.8 |
| NH₄C1 | 0.8-1.2 |

c) centrifuging the fermentation culture, obtained in step (b), followed by separation of culture fluid from pellets using filter paper (Whatman no.1) to obtain the said multienzyme system in form of culture fluids.

2. The process for the production of multienzyme system as claimed in claim 1 wherein the Aspergillus strain is selected from the group consisting of MTCC 5123, MCC0013 and MCC0014.

3. The process for the production of multienzyme system according to claim 1, wherein the multienzyme system comprises the following composition:

| Ingredients | Proportion (units) | |
|---|---|---|
| α-amylase | 6.57-15.47 | U |
| amyloglucosidase | 1.11 × 10⁵-1.21 × 10⁵ | U |
| CMCase | 15.3444.26 | U |
| pectinase | 0.04-0.76 | U |
| β-galactosidase | 0.34-1.29 | U |
| xylanase | 0.41-0.75 | U |
